(19) United States
(12) Patent Application Publication
(10) Pub. No.: US 2012/0010588 A1
(43) Pub. Date: Jan. 12, 2012

(54) FEMALE PART OF HOOK AND LOOP FASTENER

(76) Inventor: Kenichiro Morishita, Kanagawa Pref. (JP)

(21) Appl. No.: 13/061,407

(22) PCT Filed: Sep. 3, 2009
(86) PCT No.: PCT/US09/55817

§ 371 Date: Feb. 28, 2011

(30) Foreign Application Priority Data
Sep. 10, 2008 (JP) 2008 232543

(51) Int. Cl. A61F 13/62; A44B 18/00

(52) U.S. Cl. 604/391; 24/450

(57) ABSTRACT

To provide a female part of a hook and loop fastener exhibiting excellent engaging force, where the left-right difference of engaging force is small. A female part of a hook and loop fastener having a knitted fabric composed of warps, wefts and loop yarns, and a substrate, wherein a structure of the warps is a chain stitch on which open and close nodes are alternately arranged, and the loop yarns are only fixed to the close nodes of the warps with open nodes of the loop yarns, and the loop yarns project in left and right directions alternately from only one surface of the knitted fabric.

### TECHNICAL FIELD

The present invention relates to a female part of a hook and loop fastener, to a hook and loop fastener having the female part, and to an absorbent article having the hook and loop fastener.

### BACKGROUND

A hook and loop fastener is widely used for fastening flooring material, in open/close application for clothes, etc., and also for engagement of absorbent articles such as diapers because of the simplicity of the attaching/detaching operation. A hook and loop fastener is composed of a male part and a female part for engaging with it. Specifically, it is locked by engagement of loops in the female part with hooks of the male part. In the prior art, an attempt has been made to develop female material providing loops of sufficient engaging force with the hooks of male part.

In Japanese Patent Publication No. S55-107561, there is disclosed a manufacturing method of warp knitted fabric having a pattern formed by raised fabrics of different length, especially having raised fabric ribs, in which yarns forming raised fabric are engaged with nodes of the base knitting texture and then cut between the engaged nodes.

In Japanese Patent Publication No. 2000-303330, there is disclosed a woven fabric with knitted loops comprising substrate woven fabric that comprises warp wales for forming the meshes of mutually parallel wales with stitches and wefts being bound to the warp wales to form the substrate fabric, and two legs knitted with the substrate fabric, wherein the portions of wefts bound to wales are disposed between the first wale and the third wale on the mesh of the second wale and the fourth wale, and two legs of a loop are knitted with the second and the fourth stitches.

In Japanese Patent Publication No. 2004-236960, there is disclosed a female material piece for a hook-and-loop type fastener, having the structure in which it is joined to the substrate surface via a warp knitted fabric which has loops formed to receive hooks, wherein the warp knitted fabric is knitted with a loop pile knitting texture engaged with a swing knitting texture by yarns threaded through a middle guide bar and with a swing knitting texture by yarns threaded through a rear guide bar such that the directions of the loop piles adjacent in the knitting direction are alternately different to the right and left, and with a base knitting texture engaged by yarns threaded through a front guide bar with a swing knitting texture or a loop pile forming knitting texture, respectively.

### SUMMARY

It is required to provide a female part of a hook and loop fastener, which has an excellent engaging force for engaging with a male part, and in which the left-right difference of the engaging force is small, and which can be simply produced.

The present invention provides a female part of a hook and loop fastener comprising a substrate and a knitted fabric composed of warps, wefts, and loop yarns, wherein a structure of the warps is a chain stitch on which open and close nodes are alternately arranged, loop yarns are fixed to the close nodes of the warp yarns only with open nodes of the loop yarn, and the loop yarns projects in right and left direction alternately relative to a direction of the warps from only one surface of the knitted fabric.

A female part of a hook and loop fastener according to the present invention has excellent engaging force for engaging with a male part, and left-right difference of the engaging force is small. The female part of a hook and loop fastener according to the present invention can be simply produced, and can be advantageously used as a female part of a hook and loop fastener in an absorbent article such as a paper diaper.

### BRIEF DESCRIPTION OF THE DRAWINGS

The present invention will be described in detail below with reference to drawings showing an embodiment thereof. A female part of a hook and loop fastener according to the present invention is not limited to the embodiment that follows.

FIG. 1 is a schematic view showing the structure of the section of a female part of a hook and loop fastener according to an embodiment of the present invention;

FIG. 2A is a schematic view showing the knitting texture of the knitted fabric in the female part of a hook and loop fastener of an embodiment (Examples 1 and 2) of the present invention;

FIG. 2B is a schematic view showing the state of loop yarns (one loop each for left and right) formed on the knitted fabric and observed when the knitted fabric was cut along the line A-A of FIG. 2A (perpendicular to the warp direction);

FIG. 3A is a schematic view showing the pattern of knitting texture of the knitted fabric shown in FIG. 2A;

FIG. 3B is a schematic view showing the pattern of knitting texture of the knitted fabric shown in FIG. 3A, as decomposed in warps, wefts and loop yarns, showing (a) loop yarns, (b) warps, and (c) wefts;

FIG. 4 is a view useful for explaining the projecting angle θ of the loop yarns relative to the surface of the knitted fabric;

FIG. 5 is a schematic view showing the structure of the section of the hook and loop fastener according to an embodiment of the present invention;

FIG. 6A is a schematic view showing the knitting texture of the female part of the hook and loop fastener according to an embodiment (Example 3) of the present invention;

FIG. 6B is a schematic view showing the state of loop yarns (one loop each for left and right) formed on the knitted fabric and observed when the knitted fabric was cut along the line B-B of FIG. 6A (perpendicular to the warp direction);

FIG. 7 is a schematic view showing the pattern of knitting texture of the knitted fabric shown in FIG. 6A;

FIG. 8 is a schematic view showing the state of loop yarns formed on the surface of the knitted fabric of the female part of the hook and loop fastener of Comparative example 1; and

FIG. 9 is a schematic view showing the state of loop yarns formed on the surface of the knitted fabric of the female part of the hook and loop fastener of Comparative example 2.

### DETAILED DESCRIPTION

Female part 10 of a hook and loop fastener of the present invention has a knitted fabric composed of warps 21 (not shown), wefts 22 (not shown) and loop yarns 23, and substrate 30. In knitted fabric 20, loop yarns 23 form a pile of knitted fabric 20, and warps 21 and wefts 22 form base knitting texture 24, with loop yarns 23 projecting only from one surface of knitted fabric 20 alternately in the left and right direction relative to warps 21.

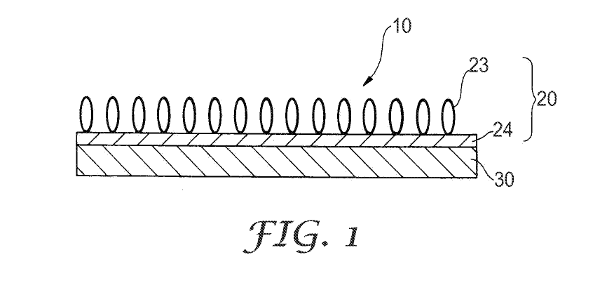

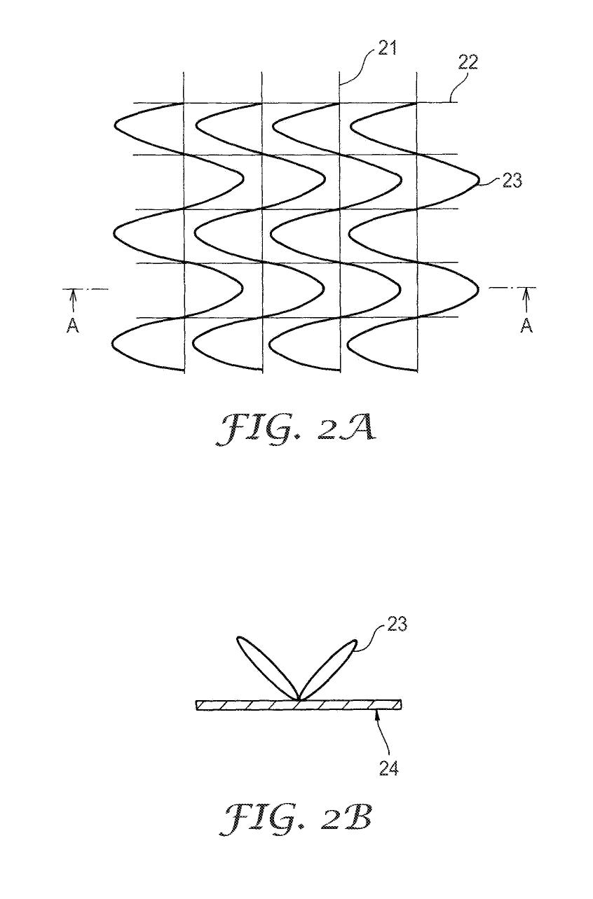

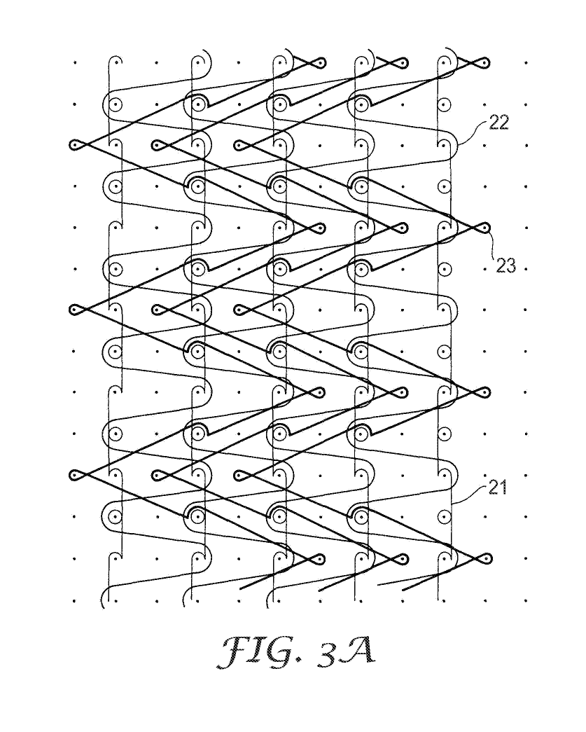

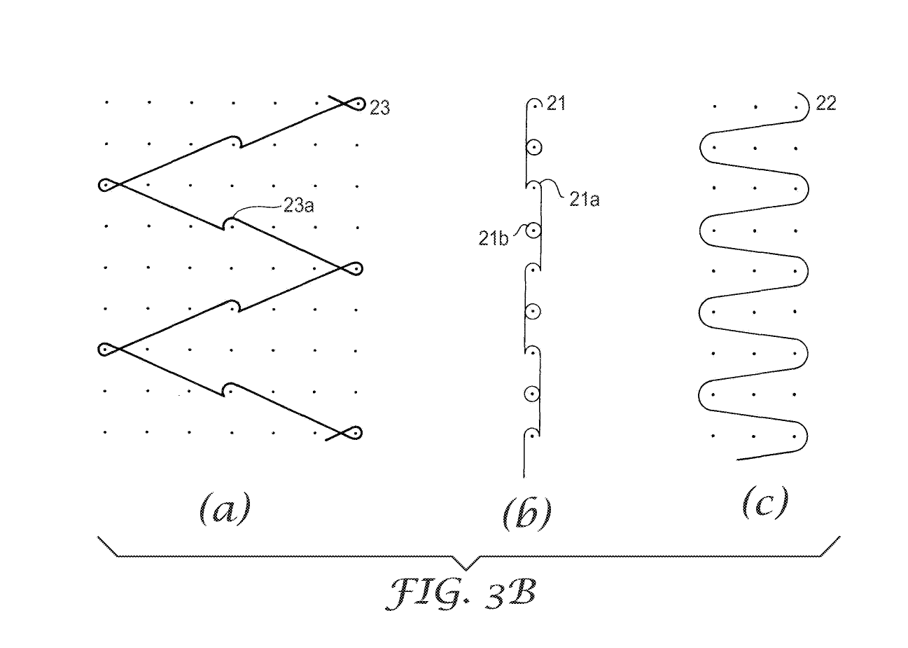

FIG. 2A is a schematic view showing the knitting texture of knitted fabric 20 having the construction of FIG. 1, FIG. 3A is a schematic view showing a pattern of the knitting texture, and FIG. 3B is a view showing the pattern of knitting texture of FIG. 3A decomposed into warps 21, wefts 22 and loop yarns 23. As shown in FIG. 3A and FIG. 3B, in knitted fabric 20, the structure of warps 21 is a chain stitch on which open nodes 21a and close nodes 21b are alternately arranged, and loop yarns 23 are fixed to warps 21 with only close nodes 21b of warps 21 engaged with open nodes 23a of loop yarns 23. Wefts 22 are fixed to warps 21 at open nodes 21a and close nodes 21b of warp 21. With such knitting texture, loop yarns 23 are constructed to project from only one surface of knitted fabric 20 alternately to the left and to the right relative to warps 21.

The knitting texture of knitted fabric 20 will be described below with reference to FIG. 3A and FIG. 3B.

Warps 21 are formed by a chain stitch on which the pattern 1-0/0-1/0-1/1-0 is repeated. As described above, with a repetition of this pattern, open node 21a and close node 21b are alternately repeated to form a chain stitch. Warps 21 become base knitting texture (middle reed) 24 of knitted fabric 20.

Loop yarns 23 are sent out to the left and to the right relative to warps 21 to form a pile (front reed) of knitted fabric 20. Thus, loop yarn 23 is first wrapped at a predetermined needle position of knitted fabric 20 from left to right by the close node or open node (close node in FIG. 3A and FIG. 3B) to be sent out to the right. Loop yarn 23 sent out to the right engages in the state of open node 23a with close node 21b of warp 21, and is sent out further to the right in knitted fabric 20. Then, at a predetermined needle position of knitted fabric 20, it is wrapped from right to left by the close node or open node (close node in FIG. 3A and FIG. 3B) to be sent out to the left. Loop yarn 23 sent out to the left engages in the state of open node 23a with close node 21b of warp 21, and is sent out further to the left. With this repetition, loop yarn 23 is projected from only one surface of knitted fabric 20 alternately to the left and to the right to form a pile.

Here, the expression “the loop yarns project from only one surface of the knitted fabric alternately to the left and to the right relative to the warp yarns” means that in knitted fabric 20, when the cross sections perpendicular to a direction of warps 21 are viewed, loop yarns 23 are formed alternately to the left and to the right while maintaining a constant angle relative to the surface of base knitting texture 24 with the engaging point with the warp as the origin. Therefore, loop yarns which are formed approximately parallel to the surface of the base knitting texture (that is, the loop yarns which lie on the surface) are not included even if the loop yarns are formed to the left and to the right relative to the warps.

The pattern of loop yarns 23 may be represented as the repetition of, for example, 1-0/3-4/6-7/4-3 (the pattern shown in FIG. 3A, the case of left and right end being close nodes), 0-1/3-4/7-6/4-3 (the case of left and right end being open nodes), 1-0/4-5/8-9/5-4 (the case of left and right end being close nodes), 0-1/4-5/9-8/5-4 (the case of left and right end being open nodes).

Wefts 22 coupled with warps 21 form base knitting texture 24 (rear reed) of knitted fabric 20. Wefts 22 are inserted into the chain stitch of warps 21, for example, as the stitch in the pattern 0-0/3-3 (the pattern shown in FIG. 3A) or 0-0/4-4, and are engaged with open nodes 21a and close nodes 21b of warps 21 at positions where left-right direction of wefts 22 changes.

In female part 10 of hook and loop fastener, loop yarns 23 project, as described above, only from one surface of the knitted fabric 20 alternately to the left and to the right relative to warps 21. In view of reducing the engaging force and the left-right difference of the engaging force, the angle of projecting loop yarns 23 relative to knitted fabric 20 is preferably 30° or more relative to the surface of knitted fabric 20, and may be 45° or more in one aspect. The upper-bound of the projecting angle has no special limitation, and the angle of projecting loop yarns 23 relative to the surface of knitted fabric 20 may be 80° or may be even 90°. The angle of projecting loop yarns 23 may be different between left and right, but in view of reducing the left-right difference of engaging force, it is preferable that the angle of projecting loop yarns 23 relative to the surface of knitted fabric 20 is nearly equal for left and right.

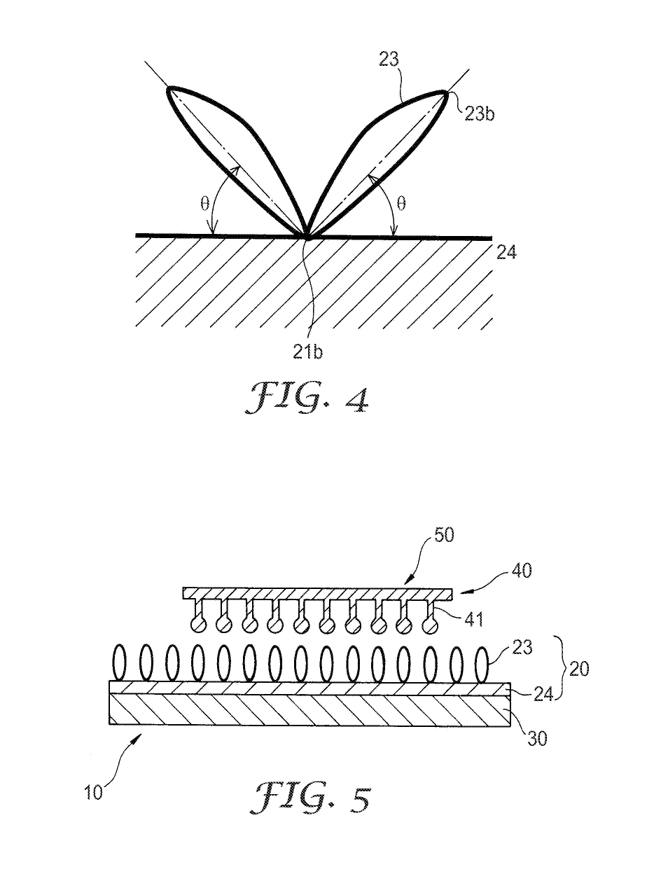

Measurement of the projecting angle of the loop yarns relative to the surface of the knitted fabric is carried out by the following method. “Projecting angle θ of the loop yarns relative to the surface of the knitted fabric” is defined, as shown in FIG. 4, as an angle formed by the tangent of the surface of the base knitting texture and the segment connecting the engaging point of loop yarn 23 with warp 21 (that is, close node 21b of warp 21) and tip 23b of loop yarn 23. First, knitted fabric 20 is cut in a direction orthogonal to a direction of warp 21 to fix base knitting texture 24 on a plane. Then, an electron microscope is used to take a photograph of the cut section of knitted fabric 20, and the projecting angle θ of the loop yarns relative to the surface of the knitted fabric is measured from the photograph.

The knitting machine used for knitting knitted fabric 20 is not particularly limited, and any knitting machine with three reeds or four reeds widely used conventionally can be used as it is. Thus, the female part of hook and loop fastener of the present invention can be simply produced at low cost.

Material of warps 21, wefts 22, and loop yarns 23 constituting knitted fabric 20 is not particularly limited, and may include polypropylene, polyester, polyethylene, polyamide, polyurethane, rayon, copolymers or mixtures thereof, and natural fiber, etc. In an aspect, in view of preventing damage of a female part due to engagement with a male part, polyamide exhibiting high strength may be used. In view of material cost and environmental stability, polyester may be used. As loop yarns, multi-filament yarns are preferred to mono-filament yarns in order to obtain higher probability for engaging with a male part. In this case, since a loop yarn of thin filament may sometimes be broken during engagement with the male part, a loop yarn of suitable diameter should be selected based on the shape, etc., of the hook and loop fastener. As regards warps and wefts, both mono-filament and multi-filament yarns may be used. In general, fineness of monofilament yarns may be in a range of 20-220 dtex, and preferably in a range of 20-100 dtex.

In one aspect, knitted fabric 20 may have basis weight of 10-100 g/m². If the basis weight is 10 g/m² or less, a shape of the knitted fabric may be difficult to be maintained when knitting, and if the basis weight is 100 g/m² or more, rigidity is increased so that, when attached to an absorbent article, flexibility may be impaired in that portion. The knitted fabric may become too dense, and when the substrate is designed aesthetically, visibility may be impaired. Since female part 10 of a hook and loop fastener of the present invention has excellent engaging force for engaging with a male part and left-right difference of the engaging force is small, the basis weight can be reduce as compared to conventional products. Therefore, a female part of a hook and loop fastener having excellent visibility can be produced at low cost.

Additional processing such as raising, embossing, printing, dyeing, coloring, and the like may be performed on female part 10 of a hook and loop fastener. Knitted fabric 20 may be dyed in all or in part. Especially when used as a member of an absorbent article such as a diaper, gloss such as luster, shining, etc., can be diminished and aesthetically excellent appearance can be obtained.

The material for substrate 30 is not particularly limited, and resin film, non-woven fabric, paper, or laminate thereof may be used. Examples of resin films include synthetic resin films such as polyester resins, for example, polypropylene, polyethylene, polystyrene, polyethylene terephthalate; polyamide resins, polyurethane resins, or laminate of these synthetic resin films. In one aspect, position indicator for engaging surface to engage with a male part, or various symbols or designs may be provided on the surface of the substrate.

Substrate 30 is joined to knitted fabric 20 on the surface opposite to the surface from which loop yarn 23 projects alternately to the left and the right. Method of joining substrate 30 to knitted fabric 20 is not particularly limited. Any method known conventionally such as dry lamination, extrusion lamination, wet lamination, thermal lamination, ultrasonic method, may be used. Among them, dry lamination method is preferable in view of productivity, flexibility, visibility of position indicator or the like provided on the substrate layer. As an adhesive for dry lamination, urethane based adhesive, EVA based adhesive, acrylic adhesive, vinyl acetate based adhesive or the like may be used. As an adhesive for wet lamination, an adhesive based on starch, casein, vinyl acetate, polyacrylic ester, or the like may be used. For extrusion lamination, resins such as polyethylene, polypropylene, or modified polyolefin may be suitably used. An adhesive or resin is not limited to those mentioned above.

In the case of dry lamination, the adhesive layer may be provided on one surface of the substrate all over the entire surface, or may be provided partially. When the adhesive layer is provided partially, there is no special restriction on the method of application. In general, an adhesive is applied in patterns, with no particular restriction on the pattern, shape, size, etc., of application. Any pattern, shape and size, for example, circle, ellipse, rectangle, polygon, etc., may be employed. When an adhesive is applied partially on the surface of the substrate, number of loop yarns caused to fall and be adhered to the surface due to seepage of adhesive through the knitted fabric can be reduced, so that decrease of engaging force for engaging with hooks of a male part can be avoided. By suitably selecting the shape of the application pattern and the amount of adhesive, the engaging force can be varied.

Female part 10 of hook and loop fastener of the present invention can be used as hook and loop fastener 50 by combining it with male part 40, as shown in FIG. 5. Here, when loop yarn 23 (pile) provided on knitted fabric 20 of female part 10 of the hook and loop fastener receives hook 41 of male part 40, female part 10 of hook and loop fastener engages with male part 40 and is adhered. In hook and loop fastener 50 having female part 10 of a hook and loop fastener of the present invention, since the female part is constructed such that loop yarns 23 project from only one surface of knitted fabric 20 alternately to the left and the right relative to warps 21, the left-right difference of the engaging force to engage with the male part tends to be suppressed.

Here, the left-right difference of engaging force can be evaluated by causing a female part of a hook and loop fastener to engage with a male part, measuring the engaging force when the two are peeled off from right to left (right engaging force) with respect to the width direction (that is, CD direction, the direction perpendicular to the warp direction), and the engaging force when the two are peeled off from left to right (left engaging force) for a plurality of points, and obtaining the absolute value of the calculated result calculated by: {(mean of the measured right engaging force)−(mean of the measured left engaging force)}/(mean of all the measured values of right engaging force and left engaging force). The nearer the value thus obtained is to zero, the smaller the left-right difference of the engaging force.

A male part may be of any type as long as satisfactory engaging force can be obtained. For example, a male part having mushroom-shaped hook, key-shaped hook, or J-shaped hook can be used. Pin density in a male part is not particularly specified, but is generally in a range of 500-5000 pins per square inch, and in one aspect, a male part having pin density of 1600 pins per square inch may be used. Material of the male part may be selected, as in the case of yarns constituting the female part, from polypropylene, polyester, polyethylene, polyamide, copolymers or mixtures thereof. Thickness of the substrate portion of the male part can be suitably set. Specifically, the hook tape commercially available from Sumitomo 3M (CS-600) etc., can be used.

The hook and loop fastener of the present invention can be used as a fastener of floor material or wall material, a fastener of clothes, a fastener for cleaning members, a fastener for car interior materials. It can also be used as a fastener of absorbent articles such as paper diapers, sanitary napkins, breastfeeding pad, etc. When the hook and loop fastener is used for an absorbent article, especially paper diaper, engaging characteristics of the female part and male part, in particular performance of the female part should be considered. In ordinary paper diapers, a pair of male parts is provided on both sides of the wearer's back, and a pair of female parts is provided on the left and right of front body for abdomen. Thus, the paper diaper has the male parts and female parts to be engaged respectively on the left and the right locations. If the left-right difference of engaging force, that is, the difference between the forces required to peel-off to the left direction and the right direction, is large, the wearer may feel anxiety in the reliable fastening function of the paper diaper provided by the hook and loop fastener. The female part of the present invention has the structure in which the loop yarns project alternately to the left and the right relative to the warps, so that the left-right difference of the engaging force for engaging with the male part is small, and therefore, is particularly suited to such application.

When the female part of hook and loop fastener of the present invention is used in a hook and loop fastener for an absorbent article, means for fastening to such absorbent article may include adhesion by means of, for example, gluing, thermal fusion, ultrasonic bonding, integral molding, mechanical fastening such as sewing, stapler, etc. For fastening by means of gluing, known adhesives such as rubber based adhesive, acryl based adhesive, silicone based adhesive, EVA based adhesive, for example, SIS, SBS, etc., may be suitably selected as required, but adhesives are not limited to those resins.

### EXAMPLES

Examples of the present invention will be described in further detail below. It is to be understood that the present invention is not limited to these Examples.

### Example 1

A knitting machine with three reeds (manufactured by Karl Mayer Co.) was used to knit a knitted fabric having knitting texture of loop yarns 1-0/3-4/6-7/4-3, warps 1-0/0-1/0-1/1-0, and wefts 0-0/3-3 in tricot pile knitting. As material for loop yarns, warps and wefts, polyamide (78 Dtex, 24 threads (manufactured by TORAY Co.)) was used for loop yarns and polyester (22 Dtex, one thread (manufactured by TEIJIN Co.) was used for warps and wefts. Knitting conditions were: 1 in 1 out (2 needle position corresponding to Wales), Course 9.6/cm, Wales 5.5/cm, basis weight was 21.8 g/cm².

The knitted fabric thus obtained was dry-laminated to a biaxial oriented polypropylene substrate (Trade name: FOR2, manufactured by Futamura Chemical Co., (corona processing on both faces)) of 12 μm in thickness, using a polyurethane adhesive to obtain a female part of a hook and loop fastener. The knitting texture of the knitted fabric of the hook and loop fastener thus obtained is schematically shown in FIG. 2A. FIG. 2B is a schematic view showing the state of the loop yarns (one loop each for left and right) formed in the knitted fabric, which is observed when the knitted fabric was cut along the line A-A of FIG. 2A (a direction perpendicular to a warp direction). The adhesive was a mixture of polyurethane main component (Trade name: TAKELAC A969v, manufactured by Mitsui Chemical Polyurethane Co.) and isocyanate curing agent (Trade name: TAKENATE A5, manufactured by Mitsui Chemical Polyurethane Co.) and silica (Trade name: TOKUSIL USA, manufactured by Tokuyama Co.), with the amount of silica in solid being 8% by weight based on total weight of the adhesive. Specifically, to a solvent containing stirred silica, polyurethane main component and isocyanate curing agent were added, and after further stirring, the mixture was laminated at 150 m/min using a dry laminator manufactured by FUJISEIKI Co. The amount of applied adhesive was about 5 g/m².

### Example 2

As in Example 1, a knitting machine with three reeds (manufactured by Karl Mayer Co.) was used to knit a knitted fabric having knitting texture of loop yarns 1-0/3-4/6-7/4-3, warps 1-0/0-1/0-1/1-0, and wefts 0-0/3-3 in tricot pile knitting. As material for loop yarns, warps and wefts, polyester (84 Dtex, 36 threads (manufactured by TORAY Co.)) was used for loop yarns and polyester (22 Dtex, one thread (manufactured by TEIJIN Co.) was used for warps and wefts. Knitting conditions were: 1 in 1 out (2 needle position corresponding to Wales), Course 10.4/cm, Wales 5.5/cm, basis weight was 24.2 g/cm². As in Example 1, a female part of a hook and loop fastener was obtained. The knitting texture of the knitted fabric of the hook and loop fastener thus obtained, and the state of the loop yarns formed into knitted fabric was the same as in Example 1.

### Example 3

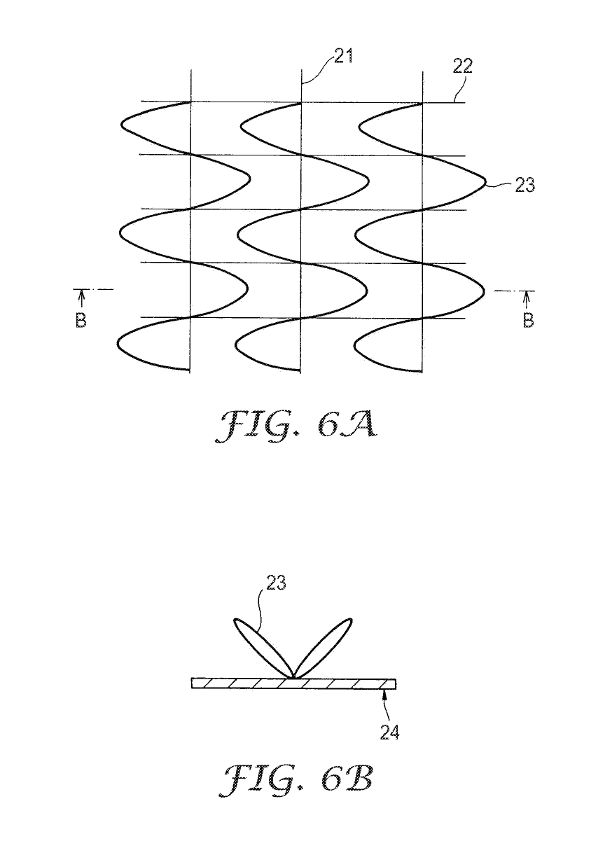

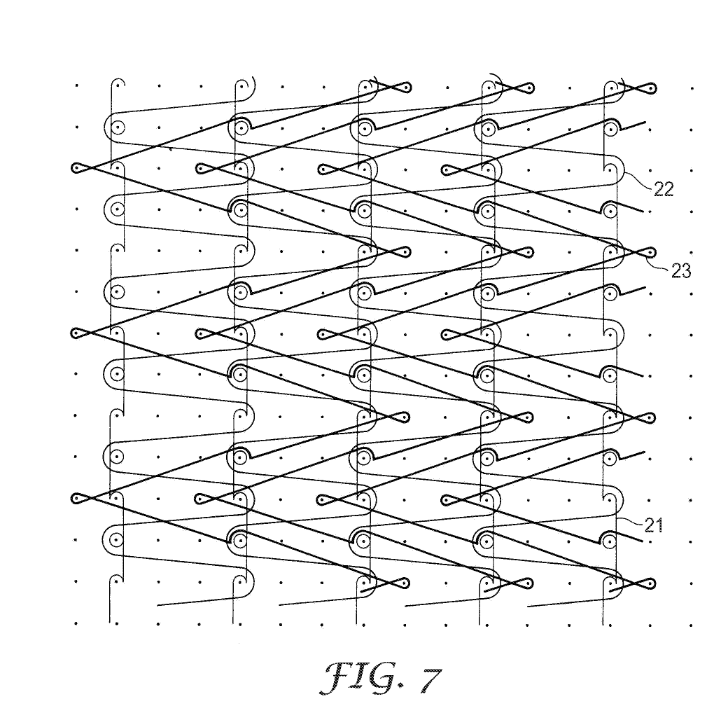

A knitting machine with three reeds (manufactured by Karl Mayer Co.) was used to knit a knitted fabric having knitting texture of loop yarns 1-0/4-5/8-9/5-4, warps 1-0/0-1/0-1/1-0, and wefts 0-0/4-4 in tricot pile knitting. As material for loop yarns, warps and wefts, polyester (84 Dtex, 36 threads (manufactured by TORAY Co.)) was used for loop yarns and polyester (22 Dtex, one thread (manufactured by TEIJIN Co.) was used for warps and wefts. Knitting conditions were: 1 in 2 out (3 needle position corresponding to Wales), Course 10.4/cm, Wales 3.7/cm, basis weight was 17.9 g/cm². As in Example 1, a female part of a hook and loop fastener was obtained. The knitting texture of the knitted fabric of the hook and loop fastener thus obtained, is schematically shown in FIG. 6A, and the pattern of this knitting texture is schematically shown in FIG. 7, respectively. FIG. 6B is a schematic view showing the state of the loop yarns (one loop each for left and right) formed in the knitted fabric, which was observed when the knitted fabric was cut along the line B-B of FIG. 6A (the direction perpendicular to the warp direction).

### Comparative Example

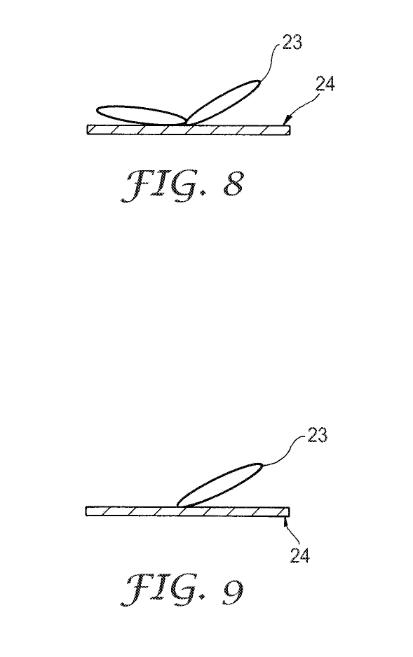

Knitted fabric made from polyamide having a basis weight of 22 g/m² was used to obtain a female part of a hook and loop fastener, as in Example 1. FIG. 8 is a schematic view showing the state of the loop yarns (one loop each for left and right) formed in the knitted fabric, which was observed when the knitted fabric was cut in a direction perpendicular to a warp direction of the knitted fabric.

### Comparative Example 2

Knitted fabric made from polyester having a basis weight of 26.5 g/m² was used to obtain a female part of a hook and loop fastener, as in Example 1. FIG. 9 is a schematic view showing the state of the loop yarns (one loop each for left and right) formed in the knitted fabric, which was observed when the knitted fabric was cut in a direction perpendicular to a warp direction of the knitted fabric. As can be seen from FIG. 9, in the hook and loop fastener of Comparative example 2, loop yarns are formed not both in the left and in the right direction, but in only one direction.

### Evaluation of a Female Part of a Hook and Loop Fastener

### Engaging Force

On the female part of the hook and loop fastener obtained in the Examples and Comparative examples described above, a male part (CS-600, manufactured by Sumitomo 3M Co.) of 25 mm in width, was placed and press bonded with 2 kg roller. Then, the female part and the male part were pulled in horizontal direction with a force of 1 kg to cause both to engage with each other. The female part and the male part of the engaged hook and loop fastener were stretched in vertical direction at stretching rate of 300 mm/min, and the peeling force at the time of peeling-off was measured to obtain the engaging force (N/25 mm). The engaging force was measured by peeling off the engaged female part of the hook and loop fastener from the male part either from right to left (right engaging force) or left to right (left engaging force) relative to the width direction (that is, CD direction, perpendicular to the warp direction), each six times. The engaging force was obtained by averaging 12 measured values. The results obtained are shown in Table 1.

### Directionality of Engaging Force

Directionality of engaging force was obtained from six measured values each of the right engaging force and the left engaging force measured as described above, using the equation: {(mean of six measured right engaging force)−(mean of six measured left engaging force)}/(mean of all (twelve) measured right engaging force and left engaging force) and expressing the result in an absolute value. The closer the value is to zero, the smaller is the left-right difference of the engaging force. Results are shown in Table 1.

Projecting angle of Loop Yarns

As shown in FIG. 4, the angle formed by the tangent of the base texture surface and the segment connecting the engaging point of loop yarn with the warp and the distal end of the loop yarn was defined as “projecting angle θ of the loop yarn relative to the surface of knitted fabric”. First, the knitted fabric was cut in a direction perpendicular to a warp direction, and the surface of base texture 24 was fixed to a plane. Then, an electron microscope was used to take photographs of the section of the knitted fabric. Projecting angle of the loop yarn relative to the surface of the knitted fabric was measured from the photograph, five points each for left and for right direction (ten points in total) (for Comparative example 2, five points only for one direction). Since in Example 1-3, there is no left-right difference in the projecting angle of loop yarns, mean value for all measured value (ten points) was calculated. For Comparative example 1, mean value each for left and right projecting angle of the loop yarns, and for Comparative example 2, mean value of the projecting angle of the loop yarns for only one direction were calculated, respectively. Results are shown in Table 1.

### Delamination Force

The knitted fabric of the female part of the hook and loop fastener and the substrate bonded thereto with an adhesive was set at 25 mm inter-chuck, was stretched in vertical direction at stretching rate of 300 mm/min, and the force at the time of separation of the knitted fabric and the substrate was measured by using a Tensilon tensile tester to obtain delamination force (N/25 mm). Results are shown in Table 1.

TABLE 1

| | | | | Compar- | Compar- |
|---|---|---|---|---|---|
| | | | | ative | ative |
| | Exam- | Exam- | Exam- | exam- | exam- |
| | ple 1 | ple 2 | ple 3 | ple 1 | example 2 |
| Basis weight (g/m²) | 21.8 | 24.2 | 17.9 | 22 | 26.5 |
| Engaging force | 2.0 | 2.8 | 1.7 | 2.0 | 2.0 |
| (N/25 mm) | | | | | |
| Directionality of | 0.07 | 0.05 | 0.09 | 0.21 | 0.56 |
| engaging force | | | | | |
| Projecting angle | 70 | 70 | 45 | 10/45 | 30* |
| of loop yarns | | | | | |
| (degrees) | | | | | |
| Delamination force | 4.3 | 4.5 | 3.4 | 3.4 | 3.3 |
| (N/25 mm) | | | | | |

*loop yarns only in one direction

1. A female part of a hook and loop fastener comprising a substrate and a knitted fabric composed of warps, wefts and loop yarns, wherein
a structure of the warps is a chain stitch on which open and close nodes are alternately arranged,
the loop yarns are only fixed to the close nodes of the warp yarns with open nodes of the loop yarn, and
the loop yarns project in right and left directions alternately in a direction of the warps, from only one surface of the knitted fabric.

2. The female part according to claim 1, wherein a projecting angle of the loop yarns from the surface of the knitted fabric is not less than 30 degree.

3. A hook and loop fastener which comprises the female part according to claim 1 and a male part.

4. An absorbent article which comprises the hook and loop fastener according to claim 3.
